SEQUENCE LISTING (9 sequences)

SEQ ID NO: 1 - DNA, 2362 bp, Ricinus communis
cctaaaaaaagtaaattactctaatcgacattatatgaattttaactaattccgtttctaatttataattatttcgttaaaccaatcaattccctttaaacactgcttatgcatattctgtctcaatttatatatggcatgcatcttccgtattaatttataagttatttttattgatcaagtatttgtggttttctttatataaaaaaatgtattagtgtttttctgtattaattttataagttcatctttatgagaatgctaatgtatttggacagccaataaaattccagaattgctgcaatcaaagatgaaaccgggaggaaatactattgtaatatggatgtatgcagtggcaacatggctttgttttggatccacctcagggtggtctttcacattagaggataacaacatattccccaaacaatacccaattataaactttaccacagcgggtgccactgtgcaaagctacacaaactttatcagagctgttcgcggtcgtttaacaactggagctgatgtgagacatgaaataccagtgttgccaaacagagttggtttgcctataaaccaacggtttattttagttgaactctcaaatcatgcagagctttctgttacattagcgctggatgtcaccaatgcatatgtggtcggctaccgtgctggaaatagcgcatatttctttcatcctgacaatcaggaagatgcagaagcaatcactcatcttttcactgatgttcaaaatcgatatacattcgcctttggaggtaattatgatagacttgaacaacttgctggtaatctgagagaaaatatcgagttgggaaatggtccactagaggaggctatctcagcgctttattattacagtactggtggcactcagcttccaactctggctcgttcctttataatttgcatccaaatgatttcagaagcagcaagattccaatatattgagggagaaatgcgcacgagaattaggtacaaccggagatctgcaccagatcctagcgtaattacacttgagaatagttgggggagactttccactgcaattcaagagtctaaccaaggagcctttgctagtccaattcaactgcaaagacgtaatggttccaaattcagtgtgtacgatgtgagtatattaatccctatcatagctctcatggtgtatagatgcgcacctccaccatcgtcacagttttctttgcttataaggccagtggtaccaaattttaatgctgatgtttgtatggatcctgagcccatagtgcgtatcgtaggtcgaaatggtctatgtgttgatgttagggatggaagattccacaacggaaacgcaatacagttgtggccatgcaagtctaatacagatgcaaatcagctctggactttgaaaagagacaatactattcgatctaatggaaagtgtttaactacttacgggtacagtccgggagtctatgtgatgatctatgattgcaatactgctgcaactgatgccacccgctggcaaatatgggataatggaaccatcataaatcccagatctagtctagttttagcagcgacatcagggaacagtggtaccacacttacagtgcaaaccaacatttatgccgttagtcaaggttggcttcctactaataatacacaaccttttgtgacaaccattgttgggctatatggtctgtgcttgcaagcaaatagtggacaagtatggatagaggactgtagcagtgaaaaggctgaacaacagtgggctctttatgcagatggttcaatacgtcctcagcaaaaccgagataattgccttacaagtgattctaatatacgggaaacagttgtcaagatcctctcttgtggccctgcatcctctggccaacgatggatgttcaagaatgatggaaccattttaaatttgtatagtgggttggtgttagatgtgagggcatcggatccgagccttaaacaaatcattctttaccctctccatggtgacccaaaccaaatatggttaccattattttgatagacagattactctcttgcagtgtgtatgtcctgctatgaaaatagatggcttaaataaaaaggacattgtaaattttgtaactgaaaggacagcaagttattgcagtccagtatctaataagagcacaactattgtcttgtgcattctaaatttatggatgaattgtatgaattaagctaattattttggtcatcagacttgatatctttttgaataaaataaataatatgttttttcaaacttataaatctatgaatgatatgaatataatgcggagactagtcaatcttttatgtaattctatgatgataaaagctt

SEQ ID NO: 2 - DNA, 27 bp, Artificial Sequence
  Description of Artificial Sequence Synthetic primer
ccctatcatagctctcatggtgtatag

SEQ ID NO: 3 - DNA, 24 bp, Artificial Sequence
  Description of Artificial Sequence Synthetic primer
acaaacatcagcattaaaatttgg

SEQ ID NO: 4 - DNA, 21 bp, Artificial Sequence
  Description of Artificial Sequence Synthetic primer
catcgtcacagttttctttgc

SEQ ID NO: 5 - DNA, 20 bp, Artificial Sequence
  Description of Artificial Sequence Synthetic primer
catttcgacctacgatacgc

SEQ ID NO: 6 - DNA, 21 bp, Artificial Sequence
  Description of Artificial Sequence Synthetic primer
ggccagtggtaccaaatttta

SEQ ID NO: 7 - DNA, 20 bp, Artificial Sequence
  Description of Artificial Sequence Synthetic primer
tcgacctacgatacgcacta

SEQ ID NO: 8 - DNA, 18 bp, Artificial Sequence
  Description of Artificial Sequence Synthetic primer
gcgcacctccaccatcgt

SEQ ID NO: 9 - DNA, 23 bp, Artificial Sequence
  Description of Artificial Sequence Synthetic primer
tgtttgtatggatcctgagccca

(19) United States
(12) Patent Application Publication
(10) Pub. No.: US 2006/0240447 A1
(43) Pub. Date: Oct. 26, 2006

(54) Oligonucleotide sequences for the detection of ricin gene and toxin

(75) Inventor: John Walter Czajka, West Grove, PA (US)

Correspondence Address: FOLEY AND LARDNER LLP;SUITE 500, 3000 K STREET NW, WASHINGTON, DC, 20007, US

(73) Assignee: SMITHS DETECTION INC. - EDGEWOOD

(21) Appl. No.: 11/295,718

(22) Filed: Dec. 7, 2005

Related U.S. Application Data
(60) Provisional application No. 60/634,098, filed on Dec. 8, 2004.

(51) Int. Cl. C12Q 1/68; C07H 21/04

(52) U.S. Cl. 435/6; 536/24.3

(57) ABSTRACT

Novel oligonucleotides useful as primers and probes in the detection and identification of ricin toxin gene and gene product are provided. A method and kit for detecting and identifying a nucleic acid from Ricinus communes are also provided.

### BACKGROUND

Ricin toxin (“ricin”), found in the bean of the castor plant, Ricinis communis, is one of the most toxic and easily produced plant-derived toxins. Ricin toxin is a likely candidate for use in a biological warfare attack because it is easily and inexpensively produced in a fairly low technology setting. Moreover, ricin toxin is very stable obviating the need for special storage conditions. Ricin toxin can be prepared in liquid or crystalline form, or it can be lyophilized to make it a dry powder. Ricin toxin can be disseminated by an enemy as an aerosol, or used as a sabotage, assassination, or terrorist weapon.

Ricin toxin, a disulfide linked heterodimer, is a ribosome-inactivating protein, which modifies the 28S subunit of eukaryotic ribosomes blocking protein synthesis. The clinical symptoms depend on the route of exposure. Aerosol exposure, which is likely in a biological warfare attack, causes fever, chest tightness, cough, dyspnea, nausea, and arthralgia. At lethal doses, severe pathophysiologic changes of the respiratory tract, including necrosis, severe alveolar flooding, lung inflammation with progressive cough, dyspnea, cyanosis, pulmonary edema, and death can occur. Ingestion of ricin toxin causes gastrointestinal hemorrhage with hepatic, splenic, and renal necrosis.

Ricin toxin acts rapidly and irreversibly and is capable of causing death or serious debilitation. Post-exposure therapy is largely supportive in nature and often ineffective. Accordingly, detection of ricin toxin contamination before exposure to individuals, such as military personnel, HAZMAT or other first responders, is essential to prevent widespread intoxication and contamination in the event of an act of biological warfare or bioterrorism attack.

In a first responder or battlefield scenario, effective detection of ricin toxin in requires the ability rapidly detect very small amounts of toxin, either directly or indirectly. Direct detection of ricin toxin involves detection of the toxin itself, while indirect detection of ricin toxin involves, for example, detection of a gene that encodes ricin toxin. Polymerase chain reaction (PCR), a method for amplifying specific sequences of nucleic acids, makes possible the rapid detection of very small quantities of specific nucleic acid sequences present in a sample. See, e.g., U.S. Pat. Nos. 4,683,195, 4,683,202, and 4,965,188. Direct detection of an amplified nucleic acid sequence by hybridization with a sequence-specific oligonucleotide probe makes possible the detection of etiologic agents, such as ricin toxin, contained in a sample, enabling rapid and sensitive diagnostic and detection assays. However, detection of agents using PCR requires oligonucleotide primers and probes capable of specifically hybridizing to the gene encoding the agent or a gene product of that gene. Thus, there is a need in the art for oligonucleotide sequences that are specific to the ricin toxin gene.

### SUMMARY

Accordingly, oligonucleotide sequences are provided which are specific to a ricin toxin gene and identify the gene based on either a nucleic acid sequence that encodes either the A or B subunit of a ricin toxin or both subunits of a ricin toxin.

One embodiment provides an wherein the oligonucleotide comprises a nucleic acid sequence selected from the group consisting of RICFP1162-27 (SEQ ID NO: 2), RICRP1264-24 (SEQ ID NO: 3), RICFP1203-21 (SEQ ID NO: 4), RICRP1304-20 (SEQ ID NO: 5), RICFP1230-21 (SEQ ID NO: 6), RICRP1304-20b (SEQ ID NO: 7) or a conservative variant thereof.

Another embodiment provides an the oligonucleotide is selected from the group consisting of RICPRB1189-20 (SEQ ID NO: 8) and RICPRB1258-23 (SEQ ID NO: 9) or a conservative variant thereof.

A further embodiment provides pair of oligonucleotide primers comprising a first primer and a second primer, wherein the first primer comprises RICFP1162-27 (SEQ ID NO: 2) and the second primer comprises RICRP1264-24 (SEQ ID NO: 3).

An additional embodiment provides a pair of oligonucleotide primers comprising a first primer and a second primer, wherein the first primer comprises RICFP1203-21 (SEQ ID NO: 4) and the second primer comprises RICRP1304-20 (SEQ ID NO: 5).

Yet another embodiment provides pair of oligonucleotide primers comprising a first primer and a second primer, wherein the first primer comprises RICFP1230-21 (SEQ ID NO: 6) and the second primer comprises RICRP1304-20b (SEQ ID NO: 7).

An further embodiment provides a set of oligonucleotides comprising a first primer, a second primer, and a probe, wherein the first primer comprises RICFP1162-27 (SEQ ID NO: 2), the second primer comprises RICRP1264-24 (SEQ ID NO: 3), and the probe comprises RICPRB1189-20 (SEQ ID NO: 8).

Still another embodiment provides a set of oligonucleotides comprising a first primer, a second primer, and a probe, wherein the first primer comprises RICFP1162-27 (SEQ ID NO: 4), the second primer comprises RICRP1264-24 (SEQ ID NO: 5), and the probe comprises RICPRB1189-20 (SEQ ID NO: 9).

A further embodiment provides a set of oligonucleotides comprising a first primer, a second primer, and a probe, wherein the first primer comprises RICFP1203-21 SEQ ID NO: 6, the second primer comprises RICRP1304-20 SEQ ID NO: 5, and the probe comprises RICPRB1258-23 SEQ ID NO: 9.

An embodiment provides a method for detecting and identifying a nucleic acid from Ricinus communis contained in a sample, wherein said method comprises mixing the sample with a polymerase chain reaction mixture comprising two oligonucleotide primers selected from the group consisting of RICFP1162-27 (SEQ ID NO: 2), RICRP1264-24 (SEQ ID NO: 3), RICFP1203-21 (SEQ ID NO: 4), RICRP1304-20 (SEQ ID NO: 5), RICFP1230-21 (SEQ ID NO: 6), and RICRP1304-20b (SEQ ID NO: 7) or a conservative variant thereof; (b) subjecting the polymerase chain reaction mixture to conditions under which the nucleic acid is amplified; and (c) detecting the presence of amplified nucleic acid sequences.

Another embodiment provides a kit comprising at least one primer selected from the group consisting of RICFP1162-27 (SEQ ID NO: 2), RICRP1264-24 (SEQ ID NO: 3), RICFP1203-21 (SEQ ID NO: 4), RICRP1304-20 (SEQ ID NO: 5), RICFP1230-21 (SEQ ID NO: 6), RICRP1304-20b (SEQ ID NO: 7) or a conservative variant thereof and at least one probe selected from the group consisting of RICPRB1189-20 (SEQ ID NO: 8) and RICPRB1258-23 (SEQ ID NO: 9) or a conservative variant thereof.

### BRIEF DESCRIPTION OF THE FIGURES

FIG. 1 depicts a consumable in the form of a strip of material. (A) is a top view perspective and (B) is a side view perspective.

FIG. 2 depicts a container housing removably connected to a plunger housing, which can be used for collecting and analyzing a specimen.

### DETAILED DESCRIPTION

The inventors have discovered which are specific for ricin toxin. These oligonucleotides can be used as primers or hybridization probes, together or individually, for nucleic acid amplification and detection for identification of the presence of the ricin toxin gene or its gene product, including the ricin toxin.

Unless indicated otherwise, all technical and scientific terms are used in a manner that conforms to common technical usage. Generally, the nomenclature of this description and the described laboratory procedures, in cell culture, molecular genetics, and nucleic acid chemistry and hybridization, respectively, are well known and commonly employed in the art. Standard techniques are used for recombinant nucleic acid methods, oligonucleotide synthesis, microbial culture, cell culture, tissue culture, transformation, transfection, transduction, analytical chemistry, organic synthetic chemistry, chemical syntheses, chemical analysis, and pharmaceutical formulation and delivery. Generally, enzymatic reactions and purification and/or isolation steps are performed according to the manufacturers' specifications. Absent an indication to the contrary, the techniques and procedures in question are performed according to conventional methodology disclosed, for example, in Sambrook et al., MOLECULAR CLONING A LABORATORY MANUAL, 2d ed. (Cold Spring Harbor Laboratory Press, 1989), and CURRENT PROTOCOLS IN MOLECULAR BIOLOGY, John Wiley & Sons, 1989).

“Sequence identify” has an art-recognized meaning and can be calculated using published techniques. See COMPUTATIONAL MOLECULAR BIOLOGY, Lesk, ed. (Oxford University Press, 1988), BIOCOMPUTING: INFORMATICS AND GENOME PROJECTS, Smith, ed. (Academic Press, 1993), COMPUTER ANALYSIS OF SEQUENCE DATA, PART I, Griffin & Griffin, eds., (Humana Press, 1994), SEQUENCE ANALYSIS IN MOLECULAR BIOLOGY, Von Heinje ed., Academic Press (1987), SEQUENCE ANALYSIS PRIMER, Gribskov & Devereux, eds. (Macmillan Stockton Press, 1991), and Carillo & Lipton, SIAM J. Applied Math. 48: 1073 (1988). Methods commonly employed to determine identity or similarity between two sequences include but are not limited to those disclosed in GUIDE To HUGE COMPUTERS, Bishop, ed., (Academic Press, 1994) and Carillo & Lipton, supra. Methods to determine identity and similarity are codified in computer programs. Preferred computer program methods to determine identity and similarity between two sequences include but are not limited to the GCG program package (Devereux et al., Nucleic Acids Research 12: 387 (1984)), BLASTP, BLASTN, FASTA (Atschul et al., J. Mol. Biol. 215:403 (1990)), and FASTDB (Brutlag et al., Comp. App. Bioscihg. 6: 237 (1990)).

One embodiment provides methods and reagents for a PCR-based detection assay capable of detecting and identifying ricin toxin. Primers and primer pairs are provided that function in a single PCR to enable the amplification of nucleic acid regions of Ricinus communis ricin toxin gene, deposited in GenBank with accession number X52908 (SEQ ID NO: 1), its gene products and conservative variants. Identification of amplified nucleic acid can then be accomplished by hybridizing the amplified product with sequence-specific oligonucleotide probes and detecting the formation of hybridized products, such as, for example, hybrid duplexes.

PCR methods, including reverse-transcriptase-mediated polymerase chain reaction, are well known and are described, for example, in Innis et al. eds., PCR PROTOCOLS: A GUIDE TO METHODS AND APPLICATIONS, Academic Press Inc. San Diego, Calif. (1990). PCR amplification products can be detected at a single time point, followed continuously by using real-time observation, endpoint analysis, or at any suitable time interval. These methods are well known in the art. For example, methods of quantitative PCR can be carried out using kits and methods that are commercially available from, for example, Applied BioSystems and Stratagene®. See also Kochanowski, Quantitative PCR Protocols (Humana Press, 1999); Innis et al., supra.; Vandesompele et al., Genome Biol. 3: RESEARCH0034 (2002); Stein, Cell Mol. Life Sci. 59: 1235 (2002).

The present primers and probes are oligonucleotides. Oligonucleotide, as used herein, is generic and can be comprised of polydeoxyribonucleotides, polyribonucleotides, and any other type of polynucleotide which is an N-glycoside of a purine or pyrimidine base, or modified purine or pyrimidine base, including double- and single-stranded DNA, as well as double- and single-stranded RNA.

Oligonucleotide primers and probes are capable of hybridizing with a target gene, in this case, a ricin toxin gene. “Hybridization” refers to the formation of a duplex structure by two single-stranded nucleic acids by complementary base pairing. Hybridization can occur between complementary nucleic acid strands or between nucleic acid strands that contain minor regions of mismatch. Conditions under which fully complementary nucleic acid strands will hybridize are referred to as “stringent hybridization conditions.” Two single-stranded nucleic acids that are complementary except for minor regions of mismatch are referred to as “substantially complementary”. Stable duplexes of substantially complementary sequences can be achieved under less stringent hybridization conditions. Those skilled in the art of nucleic acid technology can determine duplex stability empirically considering a number of variables including, for example, the length and composition of the oligonucleotides, ionic strength, and incidence and type of mismatched base pairs.

A “sequence-specific oligonucleotide” is an oligonucleotide wherein the hybridizing region is nearly completely complementary to the sequence to be detected. The use of stringent hybridization conditions allows the detection of the specific target sequence. Stringent hybridization conditions are well-known in the art and are provided, for example, in Sambrook et al., supra. Stringent conditions are sequence-dependent and will be different in different circumstances. For example, stringent conditions are selected to be about 5° C. lower than the thermal melting point (Tₘ) for a specific sequence at a defined ionic strength and pH. The Tₘ is the temperature (under defined ionic strength and pH) at which 50% of the base pairs are dissociated. Typically, stringent conditions will be those in which the salt concentration is at least about 1.5 to 6.0 mM at pH 7.0 to 8.3 and the temperature is between 40° C. and 72° C. In one embodiment, the annealing temperature is approximately 60° C.

Under some circumstances a reduction in stringency is useful. Relaxing the stringency of the hybridizing conditions allows sequence mismatches to be tolerated; the degree of mismatch tolerated can be controlled by suitable adjustment of the hybridization conditions.

In one embodiment, an oligonucleotide specifically hybridizes to a gene possessing at least 85, at least 90, at least 95, at least 96, at least 97, at least 98, or at least 99% sequence homology to the Ricinus communis ricin toxin gene, deposited in GenBank with accession number X52908 (SEQ ID NO: 1). In another embodiment, an oligonucleotide specifically hybridizes to a ricin toxin gene product.

The present primers and probes can also be used for detection of conservative variants of the gene of SEQ ID NO: 1. A “conservative variant” is a nucleotide that hybridizes under stringent conditions to an oligonucleotide probe or primer that, under comparable conditions, also binds to nucleic acid region of a gene of SEQ ID NO: 1. A conservative variant nucleotide preferably exhibits at least about a 75 percent sequence identity with its parent gene.

Oligonucleotides can be of any suitable size, which depends on many factors, including the function or use of the oligonucleotide. Oligonucleotides can be prepared by any suitable method, including, for example, cloning, enzymatic restriction of larger nucleotides, and direct chemical synthesis by a method such as the phosphotriester method of Narang et al., Meth. Enzymol. 68:90-9 (1979), the phosphodiester method of Brown et al., Meth. Enzymol. 68:109-51 (1979), the diethylphosphoramidite method of Beaucage et al., Tetrahedron Lett. 22:1859-62 (1981), and the solid support method of U.S. Pat. No. 4,458,066. A review of synthesis methods is provided in Goodchild, Bioconjugate Chemistry 1:165-87(1990).

The term “primer” refers to an oligonucleotide, whether natural or synthetic, capable of acting as an initiating point for DNA synthesis under conditions in which synthesis of a primer extension product complementary to a nucleic acid strand is induced. For example, such conditions include inclusion of four different nucleoside triphosphates and an agent for polymerization (i.e., DNA polymerase or reverse transcriptase) in an appropriate buffer and at a suitable temperature. A primer can be a single-stranded oligodeoxyribonucleotide. The length of a primer can vary and depends on the intended use of the primer. In one embodiment, a primer is less than 40 nucleotides. In another embodiment, a primer ranges from 15 to 35 nucleotides.

A primer need not reflect the exact sequence of the template, but should be sufficiently complementary to hybridize with a template. Primers can incorporate additional features which allow for the detection or immobilization of the primer, but do not alter the basic ability of the primer to act as a point of initiation of DNA synthesis.

The oligonucleotide primers and probes can be used in an amplification or polymerase chain reaction mixture. The terms “amplification reaction mixture” and “polymerase chain reaction mixture” refer to a combination of suitable reagents for carrying out a polymerase chain reaction. A reaction mixture typically consists of oligonucleotide primers, nucleotide triphosphates (dNTP), and a DNA polymerase, such as, for example, a thermostable polymerase, in a suitable buffer. A suitable polymerase is described in, for example, U.S. Pat. No. 4,889,818.

Regions of a ricin toxin gene or gene product can be specifically amplified using oligonucleotide primers having a nucleic acid sequence of SEQ ID NOs: 2-7.

Amplified nucleic acids can be detected using any suitable method. For example, in one embodiment, double-stranded DNA can be detected using non-sequence specific detection systems such as, YO-PRO®, YoYo® and SYBR® dyes, which exhibit more fluorescence when bound to double-stranded DNA, as compared to single-standed DNA.

In another embodiment, an oligonucleotide probe that is specific for a nucleic acid region, a “hybridization probe,” can be used. There are a number of different hybridization probes, however, the central feature of methods using hybridization probes is the identification of a nucleic acid present in a sample by detecting hybridization of an oligonucleotide probe to amplified target DNA.

Probes useful in nucleic acid hybridization techniques are capable of binding to a target nucleic acid of complementary sequence through one or more types of chemical bonds, usually through complementary base pairing via hydrogen bond formation. A probe may include natural bases (i.e., A, G, U, C or T) or modified bases (7-deazaguanosine, inosine, etc.). In addition, the nucleic acids can be joined by a linkage other than a phosphodiester bond, so long as it does not interfere with hybridization. Thus, probes can be peptide nucleic acids in which the constituent bases are joined by peptide bonds rather than phosphodiester linkages.

Oligonucleotide probes can be prepared by any means known in the art. Probes useful in the present invention are capable of hybridizing to a nucleotide derived from the transcript of a ricin toxin gene. The probes are preferably at least a 2, 10, 12, 14, 16, 18, 20, 22, 24, or 30 oligonucleotide fragment and can be less than 2, 1, 0.5, 0.1, or 0.05 kb in length.

An oligonucleotide probe optionally can be bound to a molecule which allows for the detection or immobilization of the probe, but does not alter the hybridization characteristics of the probe. One of skill in the art will recognize that, in general, the complement of an oligonucleotide probe is also suitable as a probe.

Oligonucleotide probes useful in the specific detection of ricin toxin include the oligonucleotide sequences provided in SEQ ID NOs: 8-9.

Probes complementary to defined nucleic acid regions of a ricin toxin gene or gene product can be synthesized chemically, generated from longer nucleotides using restriction enzymes, or can be obtained using techniques such as polymerase chain reaction (PCR). The probes can be labeled, for example, with a radioactive, biotinylated, or fluorescent tag.

In one embodiment, sequence-specific probes can employ fluorescent resonance energy transfer (FRET) between two fluorescent dyes as the means of detection. Sequence-specific oligonucleotide probes are labeled with, for example, fluorescein, rhodamine and cyanine dyes using known labeling chemistry. An acceptor dye, such as DABCYL or methyl red, can be used as an acceptor (or quencher) dye. Intercalating dyes can also be included, such as, for example, ethydium bromide or SybrGreen.

In another embodiment, gene expression is observed in solution using Q-RTPCR. Q-RTPCR relies on detection of a fluorescent signal produced proportionally during amplification of a PCR product. See Innis et al., supra. Like the traditional PCR method, this technique employs PCR oligonucleotide primers, typically 15-30 bases long, that hybridize to opposite strands and regions flanking the DNA region of interest. Additionally, a probe (e.g., TaqMan®, Applied Biosystems) is designed to hybridize to the target sequence between the forward and reverse primers traditionally used in the PCR technique. The probe is labeled at the 5′ end with a reporter fluorophore, such as 6-carboxyfluorescein (6-FAM) and a quencher fluorophore like 6-carboxy-tetramethyl-rhodamine (TAMRA). As long as the probe is intact, fluorescent energy transfer occurs which results in the absorbance of the fluorescence emission of the reporter fluorophore by the quenching fluorophore. As Taq polymerase extends the primer, however, the intrinsic 5′ to 3′ nuclease activity of Taq degrades the probe, releasing the reporter fluorophore. The increase in the fluorescence signal detected during the amplification cycle is proportional to the amount of product generated in each cycle.

In yet another embodiment, a molecular beacon, an oligonucleotide probe having a hairpin secondary structure and a donor-acceptor pair at the 5′ and 3′ end, can be generated with complementary sequences enabling the probe to bind to amplified nucleic acid regions. See, e.g. Tyagi and Kramer, Nat. Biotechnol. 14:303-8 (1996) and Tyagi et al., Nat. Biotechnol. 18:1191-6 (2000).

In an additional embodiment, an oligonucleotide probe as described in, for example, Lee et al., Anal. Chim. Acta 457:61-70 (2002), can be used to detect amplified nucleic acid regions.

One of skill in the art will recognize that the use of different detection assay labels or immobilization methods may require minor optimizations in conditions and/or probe sequences. The specific application will determine which probes are used.

Examples of primer pairs and probes useful in the detection of ricin toxin are provided in Table 1.

TABLE 1
Primers and Probes for Ricin Toxin

| | | |
|---|---|---|
| Primer set A | | |
| RICFP1162-27 | 5′ ccctatcat | SEQ ID NO:2 |
| | agctctcatg gtgtatag | |
| RICRP1264-24 | 5′ aca aac atc agc att | SEQ ID NO:3 |
| | aaa att tgg | |
| RICPRB1189-20 at | 5′ gcgcacctcc accatcgt | SEQ ID NO:8 |

Primer set A spans both the ricin toxin subunit A
and subunit B sections of the gene.

| | | |
|---|---|---|
| Primer set B | | |
| RICFP1203-21 | 5′ catcgtca cagttttctt | SEQ ID NO:4 |
| | tgc | |
| RICRP1304-20 | 5′ cat ttc gac cta cga | SEQ ID NO:5 |
| | tac gc | |
| RICPRB1258-23 | 5′ tgt ttgtatggat | SEQ ID NO:9 |
| | cctgagccca | |
| Primer set C | | |
| RICFP1230-21 | 5′ ggccagtggtaccaaattt | SEQ ID NO:6 |
| | ta | |
| RICRP1304-20b | tcgacctacgatacgcacta | SEQ ID NO:7 |
| Primer set D | | |
| RICFP1230-21 | 5′ ggccagtggtaccaaattt | SEQ ID NO:6 |
| | ta | |
| RICRP1304-20 | 5′ cat ttc gac cta cga | SEQ ID NO:5 |
| | tac gc | |
| RICPRB1258-23 | 5′ tgt ttgtatggat | SEQ ID NO:9 |
| | cctgagccca | |

A PCR reaction can further include an internal control for calibration and verification of ricin toxin gene detection. Any suitable internal control can be used. Using an internal control, a duplexed PCR assay is run and the amplification products of both the ricin toxin gene and the internal control nucleic acid are monitored.

A PCR reaction can also include primers and probes specific for other nucleic acids of interest. For example, a PCR reaction can be multiplexed to amplify and detect genes associated with other biological hazards such as Bacillus anthracis, Yersinia pestis, Francisella tularensis, B. globigii, orthopox viruses, smallpox virus, staphylococcal enterotoxin, botulinum toxin, etc.

Detection of amplification products can be conducted by continuous monitoring, i.e. real-time PCR, end-point determination, or by detecting the level of amplification products at one or more pre-determined time points.

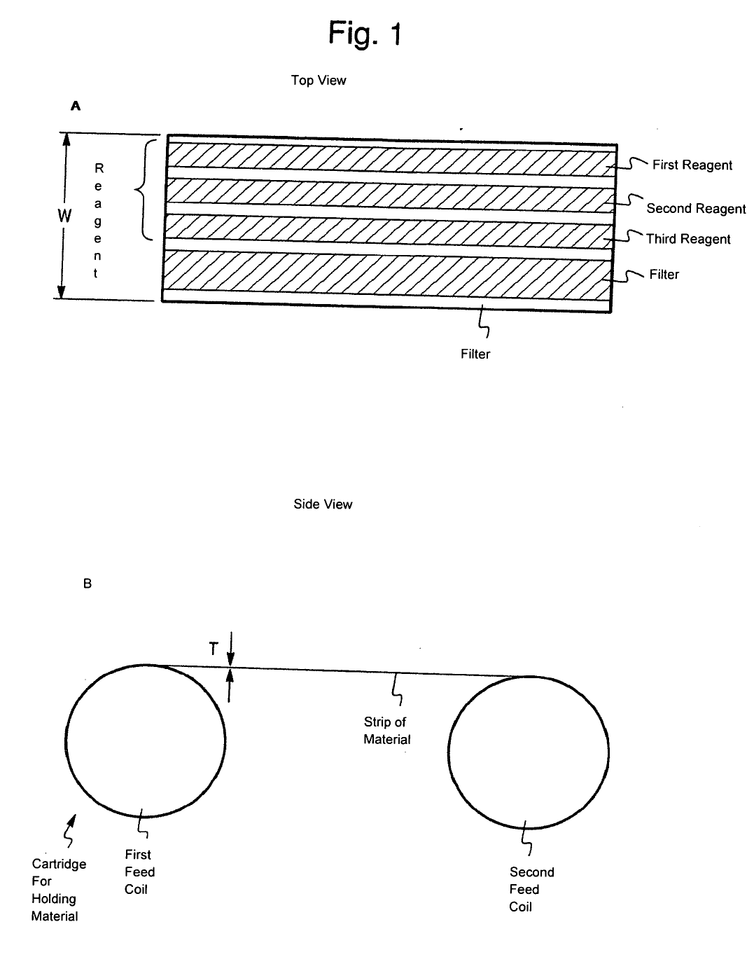

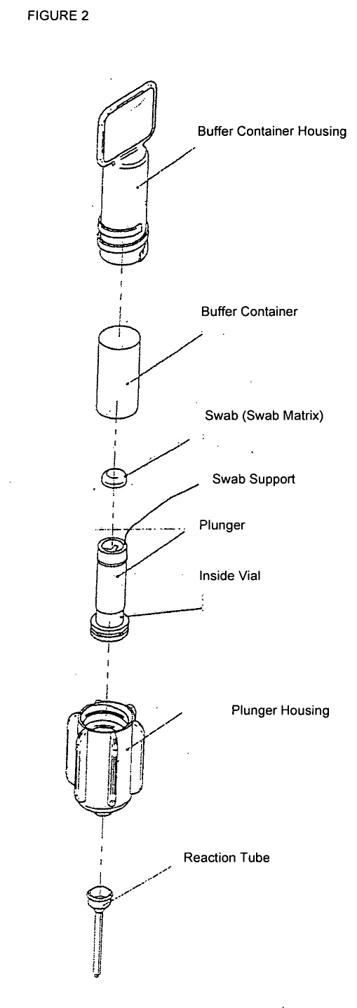

Oligonucleotides can be supplied as reagents contained in a system, such as a pre-packaged consumable. For example, a consumable can be in for form of a strip of material having a filter for capturing a biological particulate such as a nucleic acid. The strip can also contain other reagents for carrying out amplification and/or detection of a target nucleotide. The filter and the reagent can be disposed and extend longitudinally on the strip of material. See FIG. 1. In another embodiment, the oligonucleotides can be contained within a buffer container housing which is removably connected to a plunger housing. Using this device, a swab attached to an end of a plunger collects a sample of a specimen to be analyzed for biological warfare agents, such as ricin toxin. The swab and plunger are inserted into the plunger housing, a buffer container is positioned inside the buffer container housing and the buffer container housing and plunger housing are attached. A buffer passes through the swab and elutes off the sample and the sample mixes with a reagent. The prepared sample is transferred into a reaction tube by using a whipping action. See, e.g., FIG. 2.

Another embodiment comprises a kit, multicontainer unit comprising components for detecting a ricin toxin gene. A useful kit can contain a PCR reagent mixture containing a pair of primers for amplifying ricin toxin gene. Additionally, a kit can contain one or more probes specific for the ricin toxin gene. In some cases, oligonucleotide probes and/or primers can be supplied on an appropriate support membrane, such as a microwell plate, or lyophilized onto or within a consumable container, package or strip. Other optional components of a kit include, for example, an agent to catalyze the synthesis of primer extension products, substrate nucleoside triphosphates, appropriate buffers for PCR and hybridization reactions, and instructions for performing the detection method.

What is claimed is:

1. An oligonucleotide comprising a nucleic acid sequence selected from the group consisting of RICFP1162-27 (SEQ ID NO: 2), RICRP1264-24 (SEQ ID NO: 3), RICFP1203-21 (SEQ ID NO: 4), RICRP1304-20 (SEQ ID NO: 5), RICFP1230-21 (SEQ ID NO: 6), RICRP1304-20b (SEQ ID NO: 7) or a conservative variant thereof.

2. An oligonucleotide comprising a nucleic acid sequence selected from the group consisting of RICPRB1189-20 (SEQ ID NO: 8) and RICPRB1258-23 (SEQ ID NO: 9) or a conservative variant thereof.

3. A pair of oligonucleotide primers comprising a first primer and a second primer, wherein the first primer comprises RICFP1162-27 (SEQ ID NO: 2) and the second primer comprises RICRP1264-24 (SEQ ID NO: 3).

4. A pair of oligonucleotide primers comprising a first primer and a second primer, wherein the first primer comprises RICFP1203-21 (SEQ ID NO: 4) and the second primer comprises RICRP1304-20 (SEQ ID NO: 5).

5. A pair of oligonucleotide primers comprising a first primer and a second primer, wherein the first primer comprises RICFP1230-21 (SEQ ID NO: 6) and the second primer comprises RICRP1304-20b (SEQ ID NO: 7).

6. A set of oligonucleotides comprising a first primer, a second primer, and a probe, wherein the first primer comprises RICFP1162-27 (SEQ ID NO: 2), the second primer comprises RICRP1264-24 (SEQ ID NO: 3), and the probe comprises RICPRB1189-20 (SEQ ID NO: 8).

7. A set of oligonucleotides comprising a first primer, a second primer, and a probe, wherein the first primer comprises RICFP1162-27 (SEQ ID NO: 4), the second primer comprises RICRP1264-24 (SEQ ID NO: 5), and the probe comprises RICPRB1189-20 (SEQ ID NO: 9).

8. A set of oligonucleotides comprising a first primer, a second primer, and a probe, wherein the first primer comprises RICFP1203-21 SEQ ID NO: 6, the second primer comprises RICRP1304-20 SEQ ID NO: 5, and the probe comprises RICPRB1258-23 SEQ ID NO: 9.

9. A method for detecting and identifying a nucleic acid from Ricinus communis contained in a sample, wherein said method comprises:
a) mixing the sample with a polymerase chain reaction mixture comprising two oligonucleotide primers selected from the group consisting of RICFP1162-27 (SEQ ID NO: 2), RICRP1264-24 (SEQ ID NO: 3), RICFP1203-21 (SEQ ID NO: 4), RICRP1304-20 (SEQ ID NO: 5), RICFP1230-21 (SEQ ID NO: 6), and RICRP1304-20b (SEQ ID NO: 7) or a conservative variant thereof;
b) subjecting the polymerase chain reaction mixture to conditions under which the nucleic acid from is amplified; and
c) detecting the presence of amplified nucleic acid sequences.

10. The method of claim 9, wherein step (c) comprises mixing the polymerase chain reaction mixture with an oligonucleotide probe, wherein the probe is selected from the group consisting of probe is selected from the group consisting of RICPRB1189-20 (SEQ ID NO: 8) and RICPRB1258-23 (SEQ ID NO: 9) and the complements thereof, under stringent hybridization conditions, and detecting the presence of the probe hybridized to the amplified nucleic acid.

11. A kit comprising at least one primer selected from the group consisting of RICFP1162-27 (SEQ ID NO: 2), RICRP1264-24 (SEQ ID NO: 3), RICFP1203-21 (SEQ ID NO: 4), RICRP1304-20 (SEQ ID NO: 5), RICFP1230-21 (SEQ ID NO: 6), RICRP1304-20b (SEQ ID NO: 7) or a conservative variant thereof and at least one probe selected from the group consisting of RICPRB1189-20 (SEQ ID NO: 8) and RICPRB1258-23 (SEQ ID NO: 9) or a conservative variant thereof.

12. The kit of claim 11 further comprising a DNA polymerase and dNTP's.

13. The kit of claim 11 further comprising instructions.

14. The kit of claim 12 further comprising instructions.
